SEQUENCE LISTING (4 sequences)

SEQ ID NO: 1 - DNA, 21 bp, Artificial Sequence
  Synthetic primer
aacaggaaatggcctaagagg

SEQ ID NO: 2 - DNA, 6 bp, Artificial Sequence
  Synthetic primer
nccncc

SEQ ID NO: 3 - DNA, 20 bp, Artificial Sequence
  Synthetic primer
tcatggtcgacggtcagtag

SEQ ID NO: 4 - DNA, 20 bp, Artificial Sequence
  Synthetic primer
ggggaggaggtagatgccta

(19) United States
(12) Patent Application Publication
(10) Pub. No.: US 2011/0282043 A1
(43) Pub. Date: Nov. 17, 2011

(54) METHODS AND COMPOSITIONS COMPRISING NUCLEIC ACID POLYMERIZATION ENHANCERS

(75) Inventor: Dwight Dubois, Austin, TX (US)

(73) Assignee: Cenetron Diagnostics LLC, Austin, TX (US)

(21) Appl. No.: 13/046,325

(22) Filed: Mar. 11, 2011

Related U.S. Application Data
(60) Provisional application No. 61/313,431, filed on Mar. 12, 2010.

(51) Int. Cl. C12P 19/34; C07H 21/02; C12N 9/96; C07H 21/00

(52) U.S. Cl. 536/23.1; 435/188; 435/91.2; 435/91.21

(57) ABSTRACT

Embodiments of the invention are directed to compositions and methods that use non-extendable oligonucleotides to enhance or improve synthesis or amplification of nucleic acids.

This application claims priority to U.S. Provisional Patent Application 61/313,431 filed Mar. 12, 2010, which is incorporated herein by reference in its entirety.

### BACKGROUND OF THE INVENTION

I. Field of the Invention

Embodiments of this invention are directed generally to compositions and methods of use in molecular biological applications. In particular aspects the invention is directed to compositions and methods used in nucleic acid synthesis and amplification.

II. Background

Many forms of nucleic acid amplification reactions have been developed in recent years. The first method was the Polymerase Chain Reaction (PCR) which involved repeated cycles of heating to separate the DNA strands, primer annealing to the strands, and primer extension by a DNA polymerase. An alternative method for target amplification was developed called NASBA (Nucleic Acid Sequence Based Amplification) (see e.g., Compton, 1991). This method relies on the concerted action of three enzymatic activities, Reverse transcriptase, RNaseH, and RNA Polymerase, to amplify an RNA target. Still, another method has been developed which is called SDA or Strand Displacement Amplification (see e.g., Walker, 1993). The SDA method utilizes four primer sequences with two primers binding on either end of the sequence of interest. Other amplification schemes have been devised that require generating a single strand intermediate that allows primer binding for continued rounds of amplification (see e.g., Fahy et al., 1991; Guatelli et al., 1990). While the methods described above have been shown to work well, they do have some drawbacks.

Detection and analysis of variations in DNA typically involves chain extension and amplification using primers targeted for a specific sequence. The amplified DNA is then used as a target for various labeled oligonucleotide probes to identify point mutations and allelic sequence variation. If, however, the target DNA forms intra-molecular secondary structures, the DNA may not be able to hybridize with the primer or labeling probes efficiently or at all, thus resulting in no signal for the presence or absence of an SNP at the location of the secondary structure. Such intramolecular secondary structures in a single-stranded nucleic acid, such as RNA or denatured DNA, arise from the intramolecular formation of hydrogen bonds between complementary nucleotide sequences within the single-stranded nucleic acid itself. This residual secondary structure can sterically inhibit, or even block, hybrid formation between an oligonucleotide, for example a DNA or RNA oligomer being used as a primer, and its complementary sequence in the RNA or DNA.

There is a need for additional methods for increasing amplification efficiency of nucleic acids, particularly those nucleic acids with a primary structure that results in troublesome secondary structures.

### SUMMARY OF THE INVENTION

Certain aspects of the compositions and methods are directed to nucleic acids or oligonucleotides and methods of using such nucleic acids or oligonucleotides to enhance or improve synthesis or amplification of nucleic acids.

Certain embodiments include a non-extendable nucleic acid(s) or oligonucleotide(s) for enhancing or increasing the yield of nucleic acid amplification or synthesis. In further aspects, a non-extendable oligonucleotide is a nucleic acid or oligonucleotide that is not a substrate for a polymerase. A non-extendable nucleic acid or oligonucleotide will comprise 5, 6, 7, 8, 9, 10, 11, 12, 13, 14, 15, 16, 17, 18, 19, 20, 30, 40, 50 or more nucleotides or nucleotide analogs, including all ranges and values there between. In certain aspects the non-extendable oligonucleotide will comprise a G/C content of 60, 70, 80, or 95% or greater, including all values and ranges there between. In other aspects, the non-extendable oligonucleotide can comprise a nucleotide sequence of ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analog thereof, wherein x is any nucleotide or nucleotide analog. In still other aspects, the non-extendable oligonucleotide does not form a double stranded oligonucleotide by either intra-oligonucleotide or inter-oligonucleotide hybridization at 20° C. or above.

The term “non-extendable nucleic acid” or “non-extendable oligonucleotide” refers to a nucleic acid or oligonucleotide that is made non-extendable by the nature of the chemical groups at the 3′ terminus of the nucleic acid or oligonucleotide, the 5′ terminus of the nucleic acid or oligonucleotide, the 3′ position of the sugar moiety, the 5′ position of the sugar moiety, or the 3′ and 5′ position of the sugar moiety of a terminal nucleotide of the non-extendable nucleic acid or oligonucleotide, thus the nucleic acid or oligonucleotide cannot be enzymatically extended. In certain aspects, the 3′-terminus of an oligonucleotide (or other nucleic acid) can be blocked in a variety of ways using a blocking moiety. A “blocked” oligonucleotide cannot be considered a “primer.” As used herein, a “blocking moiety” is a substance used to “block” the 3′-terminus of an oligonucleotide or other nucleic acid so that it cannot be efficiently extended by a nucleic acid polymerase. A blocking moiety may be a small molecule, including, but not limited to a phosphate; a hydrogen atom; an ammonium group; a substituted or unsubstituted alkyl, aryl, heteroaryl, acyl, alkoxy, alkenyloxy, alkynyloxy, aryloxy, heteroaryl oxy group; alkamino; acylamino; or it may be a modified nucleotide, e.g., a 3′2′ dideoxynucleotide or 3′ deoxyadenosine 5′-triphosphate (cordycepin), or other modified nucleotide. Additional blocking moieties include, for example, the use of a nucleotide or a short nucleotide sequence having a 3′-to-5′ orientation, so that there is no free hydroxyl group at the 3′-terminus, the use of a 3′ alkyl group, a 3′ non-nucleotide moiety (see, e.g., Arnold et al., U.S. Pat. No. 6,031,091), phosphorothioate, alkane-diol residues, peptide nucleic acid (PNA), nucleotide residues lacking a 3′ hydroxyl group at the 3′-terminus, or a nucleic acid binding protein. Additional methods to prepare 3′-blocking oligonucleotides are well known to those of ordinary skill in the art. In certain aspects, the 5′ position in the sugar moiety of the 5′ most nucleotide can also be modified so that it is blocked from being extended.

In certain aspects, the non-extendable nucleic acid or oligonucleotide is an RNA, DNA, RNA/DNA or analog thereof. The non-extendable nucleic acid or oligonucleotide can comprise a detectable label. Detectable labels include, but are not limited to fluorescers, chemiluminescers, dyes, biotin, haptens, enzymes, enzyme substrates, enzyme cofactors, enzyme inhibitors, enzyme subunits, metal ions, electron-dense reagents, and radioactive isotopes.

Certain embodiments include methods for amplifying a target nucleic acid sequence comprising contacting the target nucleotide sequence under hybridizing conditions with (a) a nucleotide or oligonucleotide primer; (b) an amplification enhancer comprising a non-extendable nucleic acid or oligonucleotide and (c) an agent for polymerization of the nucleotides. In certain aspects the non-extendable nucleic acid or oligonucleotide comprises 5, 6, 7, 8, 9, 10, 11, 12, 13, 14, 15, 20, 40 or more nucleotides. In certain aspects the non-extendable nucleic acid or oligonucleotide has a sequence comprising ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analogs thereof, wherein x is any nucleotide or nucleotide analog. In certain aspects, the oligonucleotide does not form a double stranded oligonucleotide by either intra-oligonucleotide or inter-oligonucleotide hybridization at 20° C. or above.

In certain aspects, a target nucleic acid can be from a microbe, plant, or animal. In certain aspects a target nucleic acid is a microbial DNA or microbial RNA. In a further aspect, the target nucleic acid is a viral DNA or viral RNA.

In still further aspects, the agent for polymerization is a DNA polymerase, RNA polymerase, or nucleic acid ligase. In certain aspects, the agent for polymerization is an RNA reverse transcriptase.

Still further embodiments include methods of producing a cDNA library comprising (a) synthesizing a population of single-stranded DNA from a population of RNA molecules using: (i) an enzyme having reverse transcriptase activity, (ii) one or more oligonucleotide primers, and (iii) an amplification enhancer comprising a non-extendable nucleic acid or oligonucleotide. In certain aspects the non-extendable nucleic acid or oligonucleotide comprises 5, 6, 7, 8, 9, 10, 11, 12, 13, 14, 15, 20, 40 or more nucleotides. In certain aspects the non-extendable nucleic acid or oligonucleotide has a sequence comprising ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analogs thereof, wherein x is any nucleotide or nucleotide analog. In certain aspects, the oligonucleotide does not form or is not prone to form a double stranded oligonucleotide by either intra-oligonucleotide or inter-oligonucleotide hybridization at 20° C. or above. The method can further comprise synthesizing double-stranded cDNA from the population of single-stranded DNA generated according to step (a). The method can also comprise the step of cloning the double-stranded cDNA into a nucleic acid vector.

Certain embodiments include methods of determining a nucleic acid sequence of a target nucleic acid comprising amplifying segments of the target nucleic in the presence of a non-extendable nucleic acid or oligonucleotide. In certain aspects the non-extendable nucleic acid or oligonucleotide comprises 5, 6, 7, 8, 9, 10, 11, 12, 13, 14, 15, 20, 40 or more nucleotides. In further aspects the non-extendable nucleic acid or oligonucleotide have a sequence comprising ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analogs thereof, wherein x is any nucleotide or nucleotide analog. In certain aspects, the oligonucleotide does not form or is not prone to form a double stranded oligonucleotide by either intra-oligonucleotide or inter-oligonucleotide hybridization at 20° C. or above. The method can further comprise identifying the nucleic acid sequence of the amplified nucleic acid segments.

Other embodiments include amplicons formed by amplifying a nucleic acid in the presence of a non-extendable nucleic acid or oligonucleotide. In certain aspects the non-extendable nucleic acid or oligonucleotide comprises 5, 6, 7, 8, 9, 10, 11, 12, 13, 14, 15, 20, 40 or more nucleotides. In further aspects the non-extendable nucleic acid or oligonucleotide have a sequence comprising ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analogs thereof, wherein x is any nucleotide or nucleotide analog. In certain aspects, the oligonucleotide does not form a double stranded oligonucleotide by either intra-oligonucleotide or inter-oligonucleotide hybridization at 20° C. or above. Amplicons can range from 50; 100; 500; 1000; 5000; 10,000; 100,000 nucleobases; to 10; 100; 1,000 kilobases in length, including all values and ranges there between.

Certain embodiments include kits for amplifying nucleic acids comprising a non-extendable nucleic acid or oligonucleotide. In certain aspects the non-extendable nucleic acid or oligonucleotide comprises 5, 6, 7, 8, 9, 10, 11, 12, 13, 14, 15, 20, 40 or more nucleotides. In further aspects the non-extendable nucleic acid or oligonucleotide has a sequence comprising ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analogs thereof, wherein x is any nucleotide or nucleotide analog. In certain aspects, the oligonucleotide does not form a double stranded oligonucleotide by either intra-oligonucleotide or inter-oligonucleotide hybridization at 20° C. or above.

Still other embodiments include kits for amplifying microbial nucleic acids comprising: (a) a non-extendable nucleic acid or oligonucleotide and (b) microbe specific amplification primers. In certain aspects the non-extendable nucleic acid or oligonucleotide comprises 5, 6, 7, 8, 9, 10, 11, 12, 13, 14, 15, 20, 40 or more nucleotides. In certain aspects the non-extendable nucleic acid or oligonucleotide has a sequence comprising ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analogs thereof, wherein x is any nucleotide or nucleotide analog. In certain aspects, the oligonucleotide does not form a double stranded oligonucleotide by either intra-oligonucleotide or inter-oligonucleotide hybridization at 20° C. or above.

In certain aspects, a microbe, or pathogenic or potentially pathogenic microbe from which a nucleic acid is amplified is a virus, a bacteria, and/or a fungus. In certain aspects, a microbe is a virus. The virus can be from the Adenoviridae, Coronaviridae, Filoviridae, Flaviviridae, Hepadnaviridae, Herpesviridae, Orthomyxoviridae, Paramyxovirinae, Pneumovirinae, Picornaviridae, Poxyiridae, Retroviridae, or Togaviridae family of viruses. Virus also include HCV, HIV, HPV, Parainfluenza, Influenza, H5N1, Marburg, Ebola, Severe acute respiratory syndrome coronavirus, Yellow fever virus, Human respiratory syncytial virus, Hantavirus, or Vaccinia virus.

In yet a further aspect, the pathogenic or potentially pathogenic microbe is a bacteria. A bacteria can be an intracellular, a gram positive, or a gram negative bacteria. In a further aspect, the bacteria includes, but is not limited to a Staphylococcus, a Bacillus, a Francisella, or a Yersinia bacteria. In still a further aspect, the bacteria is Bacillus anthracis, Yersinia pestis, Francisella tularensis, Pseudomonas aeruginosa, or Staphylococcus aureas. In still a further aspect, a bacteria is a drug resistant bacteria, such as a multiple drug resistant Staphylococcus aureas (MRSA). Representative medically relevant Gram-negative bacilli include Hemophilus influenzae, Klebsiella pneumoniae, Legionella pneumophila, Pseudomonas aeruginosa, Escherichia coli, Proteus mirabilis, Enterobacter cloacae, Serratia marcescens, Helicobacter pylori, Salmonella enteritidis, and Salmonella typhii. Representative gram positive bacteria include but are not limited to Bacillus, Listeria, Staphylococcus, Streptococcus, Enterococcus, Actinobacteria, Clostridium, and Mycoplasma.

In still another aspect, the pathogenic or potentially pathogenic microbe is a fungus such as members of the family Aspergillus, Candida, Crytpococus, Histoplasma, Coccidioides, Blastomyces, Pneumocystis, or Zygomyces. In still further embodiments a fungus includes, but is not limited to Aspergillus fumigatus, Candida albicans, Cryptococcus neoformans, Histoplasma capsulatum, Coccidioides immitis, or Pneumocystis carinii. The family zygomycetes includes Basidiobolales (Basidiobolaceae), Dimargaritales (Dimargaritaceae), Endogonales (Endogonaceae), Entomophthorales (Ancylistaceae, Completoriaceae, Entomophthoraceae, Meristacraceae, Neozygitaceae), Kickxellales (Kickxellaceae), Mortierellales (Mortierellaceae), Mucorales, and Zoopagales. The family Aspergillus includes, but is not limited to Aspergillus caesiellus, A. candidus, A. carneus, A. clavatus, A. deflectus, A. flavus, A. fumigatus, A. glaucus, A. nidulans, A. niger, A. ochraceus, A. oryzae, A. parasiticus, A. penicilloides, A. restrictus, A. sojae, A. sydowi, A. tamari, A. terreus, A. ustus, A. versicolor, and the like. The family Candida includes, but is not limited to Candida albicans, C. dubliniensis, C. glabrata, C. guilliermondii, C. kefyr, C. krusei, C. lusitaniae, C. milleri, C. oleophila, C. parapsilosis, C. tropicalis, C. utilis, and the like.

Certain embodiments are directed to kits for determining the genotype of an individual, comprising (a) a non-extendable nucleic acid or oligonucleotide and (b) an allele specific hybridization (ASH) probe. In certain aspects the non-extendable nucleic acid or oligonucleotide comprises 5, 6, 7, 8, 9, 10, 11, 12, 13, 14, 15, 20, 40 or more nucleotides. In certain aspects the non-extendable nucleic acid or oligonucleotide have a sequence comprising ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analogs thereof, wherein x is any nucleotide or nucleotide analog. In certain aspects, the oligonucleotide does not form a double stranded oligonucleotide by either intra-oligonucleotide or inter-oligonucleotide hybridization at 20° C. or above.

The term “nucleic acid” is intended to encompass a singular “nucleic acid” as well as plural “nucleic acids,” and refers to any chain of two or more nucleotides, nucleosides, or nucleobases (e.g., deoxyribonucleotides or ribonucleotides) covalently bonded together. Nucleic acids include, but are not limited to, viral genomes, or portions thereof, either DNA or RNA, bacterial genomes, or portions thereof, fungal, plant or animal genomes, or portions thereof, messenger RNA (mRNA), ribosomal RNA (rRNA), transfer RNA (tRNA), plasmid DNA, mitochondrial DNA, or synthetic DNA or RNA. A nucleic acid may be provided in a linear (e.g., mRNA), circular (e.g., plasmid), or branched form, as well as a double-stranded or single-stranded form. Nucleic acids may include modified bases to alter the function or behavior of the nucleic acid, e.g., addition of a 3′-terminal dideoxynucleotide to block additional nucleotides from being added to the nucleic acid. As used herein, a “sequence” of a nucleic acid refers to the sequence of bases that make up a nucleic acid. The term “polynucleotide” may be used herein to denote a nucleic acid chain. Throughout this application, nucleic acids are designated as having a 5′-terminus and a 3′-terminus. Standard nucleic acids, e.g., DNA and RNA, are typically synthesized “3′-to-5′,” i.e., by the addition of nucleotides to the 5′-terminus of a growing nucleic acid.

A “nucleotide” is a subunit of a nucleic acid consisting of a phosphate group, a 5-carbon sugar and a nitrogenous base. The 5-carbon sugar found in RNA is ribose. In DNA, the 5-carbon sugar is 2′-deoxyribose. The term also includes analogs of such subunits, such as a methoxy group at the 2′ position of the ribose (2′-O-Me) and the like.

The term “amplifying” refers to a process whereby a portion of a nucleic acid is replicated. Unless specifically stated “amplifying” or “copying” may refer to a single replication or arithmetic, logarithmic, or exponential amplification.

The terms “amplicon” and “amplification product” refer to a nucleic acid molecule generated during an amplification procedure that is substantially complementary or identical to a sequence contained within the target nucleic acid.

As used herein, the term “oligonucleotide” or “oligo” or “oligomer” is intended to encompass a singular “oligonucleotide” as well as plural “oligonucleotides,” and refers to any polymer of two or more of nucleotides, nucleosides, nucleobases or related compounds used as a reagent in the amplification methods of the present invention, as well as subsequent detection methods. Oligonucleotide can comprise up to 100 nucleobases or less. The oligonucleotide may be DNA and/or RNA and/or analogs thereof. The term oligonucleotide does not denote any particular function to the reagent; rather, it is used generically to cover all such reagents described herein. An oligonucleotide may serve various different functions, e.g., target capture oligomers hybridize to target nucleic acids for capture and isolation of nucleic acids; or amplification oligomer include heterologous amplification oligomers, primer oligomers and promoter-based amplification oligomers.

The term “detecting” refers to quantitatively or qualitatively determining the presence or absence of an analyte, such as a nucleic acid.

The term “detectable moiety” refers to a moiety that is attached through covalent or non-covalent means to the non-target antisense primer or said non-target sense-primer. A “detectable moiety” can be a radioactive moiety, a fluorescent moiety, a chemiluminescent moiety, an antibody moiety, etc.

“Double-stranded DNA” refers to a duplex of two complementary DNA strands which by convention is drawn as a double line with a sense strand from 5′ to 3′ as the top strand and an antisense strand from 3′ to 5′ as the bottom strand.

As used herein, a “pathogen” or “microbe” is a bioagent which causes a disease or disorder.

The term “polymerase” refers to an enzyme having the ability to synthesize a complementary strand of nucleic acid from a starting template nucleic acid strand and free nucleotide triphosphates.

The term “polymerization agent” refers to any agent capable of facilitating the addition of nucleoside triphosphates to an oligonucleotide. Preferred polymerization agents are DNA and RNA polymerases.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Digital image of an agarose gel electrophoretic fractionation of amplicons produced from a RT-PCR amplification of the NS5b region of the HCV genome (nucleotide positions 7551 to 9368, based on H77 HCV reference sequence). Lane 1: cDNA synthesis and amplification in the presence of 1 μM of 3′-blocked RNA oligo (sequence: NCCNCC (SEQ ID NO:2)). Lane 2: cDNA synthesis and amplification in the presence of 0.5 μM of 3′-blocked RNA oligo (sequence: NCCNCC). Lane 3: cDNA synthesis and amplification in the absence of 3′-blocked RNA oligo (sequence: NCCNCC). Amplicon is 1818 basepairs in length. DNA Ladder: 10 kB, 8 kB, 6 kB, 5 kB, 4 kB, 3 kB, 2 kB, 1.5 kB, 1 kB, and 0.5 kB. N=equimolar mixture of A, G, T, and C.

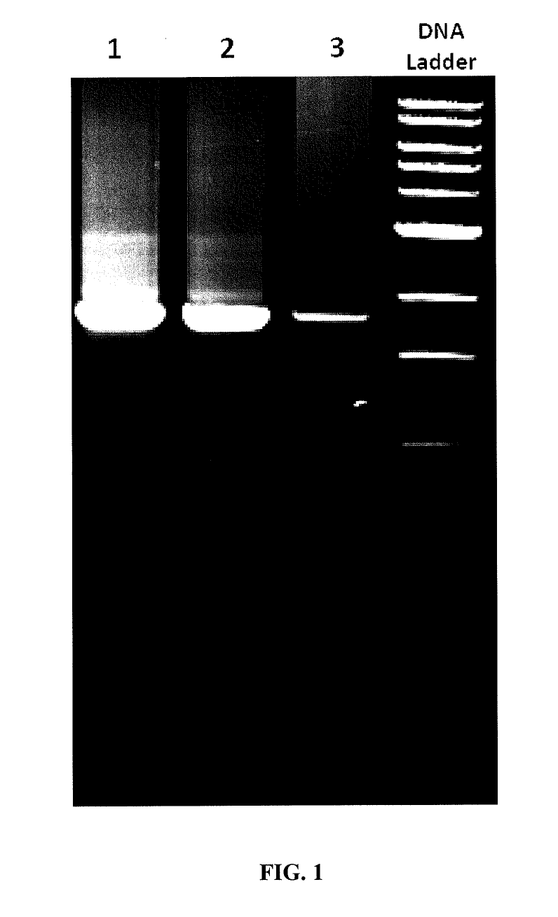

### DETAILED DESCRIPTION OF THE INVENTION

Certain embodiments include a non-extendable nucleic acid or oligonucleotide for enhancing or increasing the yield of nucleic acid amplification or synthesis. A non-extendable oligonucleotide is an oligonucleotide that is not a substrate for a polymerase or ligase. The term “non-extendable oligonucleotide” refers to an oligonucleotide that is made non-extendable by modifying the chemical groups at the 3′ position of the sugar moiety, the 5′ position of the sugar moiety, or the 3′ and 5′ position of the sugar moiety of a terminal nucleotide of the non-extendable oligonucleotide, thus the oligonucleotide cannot be enzymatically extended. In certain aspects, the 3′-terminus of an oligonucleotide (or other nucleic acid) can be blocked in a variety of ways using a blocking moiety. A “blocked” oligonucleotide cannot be considered a “primer.” As used herein, a “blocking moiety” is a substance used to “block” the 3′-terminus of an oligonucleotide or other nucleic acid so that it cannot be efficiently extended by a nucleic acid polymerase. A blocking moiety may be a small molecule, e.g., a phosphate, a hydrogen, an ammonium group, an alkyl group, an aryl group, or it may be a modified nucleotide, e.g., a 3′2′ dideoxynucleotide or 3′ deoxyadenosine 5′-triphosphate (cordycepin), or other modified nucleotide. Additional blocking moieties include, for example, the use of a nucleotide or a short nucleotide sequence having a 3′-to-5′ orientation, so that there is no free hydroxyl group at the 3′-terminus, the use of a 3′ alkyl group, a 3′ non-nucleotide moiety (see, e.g., Arnold et al., U.S. Pat. No. 6,031,091), phosphorothioate, alkane-diol residues, peptide nucleic acid (PNA), nucleotide residues lacking a 3′ hydroxyl group at the 3′-terminus, or a nucleic acid binding protein. Additional methods to prepare 3′-blocking oligonucleotides are well known to those of ordinary skill in the art.

### I. Non-Extendable Oligonucleotides

A non-extendable oligonucleotide may comprise at least one modified base moiety that is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl)uracil, and 2,6-diaminopurine.

A non-extendable oligonucleotide can also include at least one modified sugar moiety selected from the group including, but not limited to, arabinose, 2-fluoroarabinose, xylulose, and hexose.

Furthermore, a non-extendable oligonucleotide can include at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

A non-extendable oligonucleotide may be obtained by synthesis using standard methods known in the art, for example, by use of an automated DNA synthesizer (such as are commercially available from Biosearch, Applied Biosystems, etc.) and standard phosphoramidite chemistry. As examples, phosphorothioate oligonucleotides may be synthesized by the method of Stein et al. (1988) and methylphosphonate oligonucleotides can be prepared by use of controlled pore glass polymer supports (Sarin et al., 1988). Once the desired oligonucleotide is synthesized, it is cleaved from the solid support on which it was synthesized and treated by methods known in the art to remove any protecting groups present, if desired. The oligonucleotide may then be purified by any method known in the art, including extraction and gel purification. The concentration and purity of the oligonucleotide may be determined by examining an oligonucleotide that has been separated on an acrylamide gel or by measuring the optical density at 260 nm in a spectrophotometer.

### II. Nucleic Acid Synthesis and Amplification

In certain embodiments, methods can be used to synthesize or amplify a variety of nucleic acids, including, but not limited to genomic nucleic acids, coding regions of mRNAs, introns, alternatively spliced forms of a gene, non-coding RNAs that regulate gene expression and the like. Non-limiting examples of such methods is provided below.

A. Reaction Components

The following reaction components can be used in methods that involve the synthesis and/or amplification of nucleic acids.

1. Oligonucleotide Primers

The oligonucleotides are typically used as primers for synthesis and/or amplification of nucleic acids, as well as probes designed to detect amplification products. The oligonucleotides can be chemically synthesized and may be labeled with radioisotopes, chemiluminescent moieties, or fluorescent moieties in a covalent or non-covalent manner. Such labels are useful for the characterization and detection of amplification products.

2. Buffer

Buffers are typically employed to maintain a proper pH and provide the appropriate chemical conditions for synthesis and/or amplification. Buffers that may be employed are borate, phosphate, carbonate, barbital, Tris based buffers and the like. See U.S. Pat. No. 5,508,178. The pH of the reaction should be maintained in the range of about 4.5 to about 9.5, but may vary depending on the particular enzyme or method used for polymerization or synthesis. See U.S. Pat. No. 5,508,178. A standard buffer used in amplification reactions is a Tris based buffer between 10 to 150 mM, including all values and ranges there between, with a pH of around 7.5 to 8.8.

3. Salt Concentration

The concentration of salt present in the reaction can affect the ability of primers to anneal to the target nucleic acid. Potassium chloride can be added up to a concentration of about 0.1 mM to 50 mM, including all values and ranges there between, to the reaction mixture to promote primer annealing. Sodium chloride can also be added to promote primer annealing.

4. Magnesium and Manganese Ion Concentration

The concentration of magnesium ion in the reaction can also influence synthesis and amplification of nucleic acids. Primer annealing, strand denaturation, amplification specificity, primer-dimer formation, and enzyme activity are all examples of parameters that are affected by magnesium concentration. Amplification reactions can contain at least, at most, or about 2.5 to 30 mM magnesium, including all values and ranges there between, concentration excess over the concentration of dNTPs. The presence of magnesium chelators in the reaction can affect the optimal magnesium concentration. Those of skill in the art, can readily carry out a series of amplification reactions over a range of magnesium concentrations to determine the optimal magnesium concentration. The optimal magnesium concentration can vary depending on the nature of the target nucleic acid(s) and the primers being used, among other parameters.

The presence of manganese ions can also influence the synthesis and amplification reactions. The manganese ions are typically provided in the form of a salt, e.g., manganese chloride. In preferred embodiments, the Mn⁺⁺ is present in a concentration of between 1 μM to 30 mM, including all values and ranges there between. One of skill in the art can optimize the manganese ion concentration for a particular set of reaction conditions and substrates.

5. Deoxyribonucleotide and Ribonucleotide Triphosphate Concentration

Deoxyribonucleotide triphosphates (dNTPs) are added to the reaction to a final concentration of about 200 μM to about 5 mM. Each of the four dNTPs (G, A, C, T) are typically provided at equivalent concentrations. The dNTPs can be prepared from commercially available stock solutions or from dry powder stocks of each dNTP. In certain reactions the dNTPs are present at a concentration range between 1 and 10 mM, including all values and ranges there between. Ribonucleotide triphosphates (rNTPs) are added to the reaction to a final concentration of about 200 μM to about 5 mM, including all values and ranges there between.

6. Other Agents

Stabilizing agents such as gelatin, bovine serum albumin, and non-ionic detergents (e.g., Tween-20) can be added to amplification reactions.

7. Temperature

The temperature of a reaction mixture for the synthesis or amplification of a nucleic acid can vary over the range at which the enzymes or chemical reactions in the mixture are active and products are produced. For example, the methods can be carried out at constant or variable temperatures between 0, 10, 20, 30, 40, 50, 60° C. to 50, 60, 70, 80, 90, 100° C. or more, including all values and ranges there between.

8. Reaction Steps

The methods may be carried out in a discontinuous manner. That is, one or more of the synthesis or amplification steps can be performed separately and the product used as the basis of the next step. In certain embodiments, the synthesis or amplification of a nucleic acid is carried out in a single reaction vessel. Thus, typically in a single reaction vessel the reaction buffer, the nucleic acid template, the enzymes, and amplification primers are combined in a solution. In certain embodiments, a reaction can be carried out in a thermal cycler or similar machine to facilitate incubation times at one or desired temperatures.

B. Detection of the Amplification Products

Those of skill in the art will recognize that there are many ways to detect nucleic acids. The following are examples of methods used to detect nucleic acids that can be used in conjunction with the present invention. The methods can involve detecting the synthesis or amplification products of the methods described herein. These products may be detected by the use of oligonucleotides that are labeled with a detectable moiety and are incorporated into a reaction product. Alternatively, amplification products can be detected by hybridizing a detection oligonucleotide comprising a detectable moiety to an amplification product. The presence of a detectable moiety can be ascertained using appropriate means, e.g., visual means for detectable moieties producing a visible signal, a fluorometer for fluorescent labels, a spectrophotometer for labels of the visible light range, a scintillation counter for radioactive labels, etc. In addition, the following methods, as well as other methods known in the art, may be used to detect amplification products of the present invention.

1. Ethidium Bromide Staining

The method of using ethidium bromide, and other nucleic acid binding labels, to detect nucleic acids in agarose gels is well known in the art. See, e.g., Ausubel et al. Briefly, the amplification products can be electrophoresed on an agarose gel. The agarose gel is then incubated with the intercalating agent, e.g., ethidium bromide. The ethidium bromide soaked gel can then be illuminated with ultraviolet light. The ethidium bromide fluoresces under ultraviolet light and permits the visualization of DNA bands in the gel. The molecular size of the product can be estimated by co-electrophoresing a sample with known molecular sizes of nucleic acid, a “nucleic acid ladder.” Such ladders are available from a variety of commercial vendors.

2. Fluorescence Resonance Energy Transfer

Methods employing the technique of fluorescence resonance energy transfer (FRET) can be employed using the methods and compositions of the present invention. FRET is a distance-dependent interaction between a donor and acceptor molecule. The donor and acceptor molecules are fluorophores. If the fluorophores have excitation and emission spectra that overlap, then in close proximity (typically around 10-100 angstroms) the excitation of the donor fluorophore is transferred to the acceptor fluorophore.

In one particular method employing FRET, fluorescent energy transfer labels are incorporated into a primer that can adopt a hairpin structure. See U.S. Pat. Nos. 5,866,336; 5,958,700; and 5,925,517. The primers can be designed in such a manner that only when the primer adopts a linear structure, i.e., is incorporated into an amplification product, is a fluorescent signal generated.

3. TaqMan Assay

The products can be detected in solution using a fluorogenic 5′ nuclease assay—The TaqMan assay. See Holland et al. (1991); U.S. Pat. Nos. 5,538,848; 5,723,591; and 5,876,930. The TaqMan probe is designed to hybridize to a sequence within an amplification product. The 5′ end of the TaqMan probe contains a fluorescent reporter dye. The 3′ end of the probe is blocked to prevent probe extension and contains a dye that will quench the fluorescence of the 5′ fluorophore. During subsequent amplification, the 5′ fluorescent label is cleaved off if a polymerase with 5′ exonuclease activity is present in the reaction. The excising of the 5′ fluorophore results in an increase in fluorescence which can be detected.

C. Whole Genome Amplification (WGA)

In many fields of research such as genetic diagnosis, cancer research or forensic medicine, the scarcity of genomic DNA can be a severely limiting factor on the type and quantity of genetic tests that can be performed on a sample. One approach designed to overcome this problem is whole genome amplification. The objective is to amplify a limited DNA sample in a non-specific manner in order to generate a new sample that is indistinguishable from the original but with a higher DNA concentration. The aim of a typical whole genome amplification technique would be to amplify a sample up to a microgram level while respecting the original sequence representation.

A number of methods have been developed for exponential amplification of small amounts of nucleic acids, which can be performed in situ (in a background of a matrix, such as low melt agarose). These include a variety of methods of whole genome amplification (WGA), e.g., the isothermal amplification method, multiple displacement amplification (MDA). In one form of this method, two sets of primers are used that are complementary to opposite strands of nucleotide sequences flanking a target sequence. Amplification proceeds by replication initiated at each primer and continuing through the target nucleic acid sequence, with the growing strands encountering and displacing previously replicated strands. In another form of the method, a random set of primers is used to randomly prime a sample of genomic nucleic acid. The primers in the set are collectively, and randomly, complementary to nucleic acid sequences distributed throughout nucleic acid in the sample. Amplification proceeds by replication initiating at each primer and continuing so that the growing strands encounter and displace adjacent replicated strands.

Other suitable methods of whole genome amplification of small amounts of nucleic acid include, but are not limited to, ligation-mediated PCR (LMP PCR) (Tanabe et al., 2003), such as OmniPlex technology (Rubicon, Inc.), which takes fragmented genomic DNA (4-5 ng) followed by ligation of universal adapters and then amplifies using universal primers (Langmore, 2002); degenerate oligonucleotide primed PCR (DOP-PCR), which uses random primers to amplify, via PCR, genomic DNA (Telenius et al., 1992); and T7-based linear amplification of DNA (TLAD), in which a polyT tail is added to the 3′ end of fragmented genomic DNA, which then provides a binding site for a T7 promoter with a poly A tail at the 3′ end, and second strand synthesis is then performed followed by in vitro transcription using T7 polymerase in an isothermal reaction (Liu et al., 2008).

Subsequent to initial amplification by a WGA method (e.g., about 10-20, for example about 15, minutes of amplification), one can also employ additional amplification methods in which the enzymes are not as processive, such as the polymerase chain reaction (PCR), ligase chain reaction (LCR), self-sustained sequence replication (SSR), nucleic acid sequence based amplification (NASBA), strand displacement amplification (SDA), and amplification with Q-beta replicase (see, e.g., Birkenmeyer et al., 1991 and Landegren, 1993).

Following in situ amplification of the nucleic acid, the amplified nucleic acid can be visualized (e.g. by EFM), if necessary, excised (e.g. by physical dissection), separated from the agarose by treating with agarase, and purified with a conventional phenol/chloroform/ethanol procedure.

D. Enzymes

1. DNA Polymerases

In certain aspects the methods may utilize a DNA polymerase. A DNA polymerase can include, but is not limited to Taq DNA polymerase, Klenow(exo-) DNA polymerase, Bst DNA polymerase, VENT® (exo-) DNA polymerase (DNA polymerase A cloned from Thermococcus litoralis and containing the D141A and E143A mutations), Pfu(exo-) DNA polymerase, and DEEPVENT™ (exo-) DNA polymerase (DNA polymerase A, cloned from the Pyrococcus species GB-D, and containing the D141A and E143A mutations), AMPLITAQ® DNA polymerase, FS (Taq DNA polymerase that contains the G46D and F667Y mutations), THERMOSEQUENASE™ DNA polymerase (Taq DNA polymerase that contains the F667Y mutation), THERMOSEQUENASE™ II DNA polymerase (blend of THERMOSEQUENASE™ DNA polymerase and T. acidophilum pyrophosphatase), THERMINATOR™ DNA polymerase (DNA polymerase A, cloned from the Thermococcus species 9°N-7 and containing the D141A, E143A and A485L mutations), THERMINATOR™ II DNA polymerase (THERMINATOR™ DNA polymerase that contains the additional Y409V mutation), and VENT® (exo-) A488L DNA polymerase (VENT® (exo-) DNA polymerase that contains the A488L mutation).

2. RNA Polymerases

RNA polymerases (RNAPs) are used in certain aspects of the present methods for, among other things, transcribing substrates in order to provide transcripts that are part of amplification cycle. Typically, RNAPs utilize ribonucleotides and cannot utilize deoxyribonucleotides. The RNAPs can be obtained from many sources, including from prokaryotes, phage, bacteriophage, eukaryotes, fungi, plants, archaebacteria, etc. The RNAPs should be stable and active under the conditions of the amplification methods.

Examples of phage-encoded RNAPs include, without limitation, a SP6 RNAP (e.g., GenBank Accession No. Y00105), a T7 RNAP (e.g., GenBank Accession No. M38308), a T3 RNAP (e.g., GenBank Accession No X02981), and a K11 RNAP (e.g., GenBank Accession No. X53238; (Dietz et al., 1990). These phagemid RNAPs have been cloned and expressed in bacteria and several are commercially available (e.g., SP6 RNAP, T7 RNAP, T3 RNAP). For example, the T7 RNAP (Davanloo et al., 1984) and the K11 RNAP (Han et al., 1999) have been expressed as a soluble proteins in E. coli.

### III. Kits

The methods described herein may be made more convenient by using a kit format. The kit may contain all of the components necessary to perform various molecular biological methods along with instructions. For example, a kit may contain one or more non-extendable oligonucleotides, a polymerase, a reverse transcriptase, a dNTP mix, a rNTP mix, a reaction buffer, primers, control primers and control templates, and such. The kits of the invention may be designed for synthesis, amplification, or detection of nucleic acid(s), for example, RNAs expressed in a cell or tissue, or DNA or RNA from microbial genomes.

In certain embodiments, the kits can comprise one or more oligonucleotide primers that may be used to synthesize, amplify, and/or detect a nucleic acid target(s).

In some embodiments of the present invention, the kit may further comprise one or more of the following components: a reverse transcriptase enzyme, a DNA polymerase enzyme, a DNA ligase enzyme, an RNase H enzyme, a Tris buffer, a potassium salt (e.g., potassium chloride), a magnesium salt (e.g., magnesium chloride), an ammonium salt (e.g., ammonium sulfate), a reducing agent (e.g., dithiothreitol), deoxynucleoside triphosphates (dNTPs), ribonucleotide triphosphates (rNTPs), and a ribonuclease inhibitor(s). For example, the kit may include components optimized for first strand cDNA synthesis, such as a reverse transcriptase with reduced RNase H activity and increased thermal stability (e.g., SuperScript™ III Reverse Transcriptase, Invitrogen), and a dNTP stock solution to provide a final concentration of dNTPs in the range of from 50 to 5000 mM.

In various embodiments, the kit may include a detection reagent such as SYBR green dye or BEBO dye that preferentially or exclusively binds to double-stranded DNA. In other embodiments, the kit may include a forward and/or reverse primer that includes a fluorophore and quencher.

A kit of the invention can also provide reagents for in vitro transcription of cDNAs. For example, in some embodiments the kit may further include one or more of the following components: a RNA polymerase enzyme, an IPPase (Inositol polyphosphate 1-phosphatase) enzyme, a transcription buffer, a Tris buffer, a sodium salt (e.g., sodium chloride), a magnesium salt (e.g., magnesium chloride), spermidine, a reducing agent (e.g., dithiothreitol), and nucleoside triphosphates (ATP, CTP, GTP, UTP).

In another embodiment, the kit may include reagents for labeling nucleic acid products with Cy3 or Cy5 dye.

In another embodiment, the kit may include one or more of the following reagents for sequencing PCR products: Taq DNA Polymerase, T4 Polynucleotide kinase, Exonuclease I (E. coli), sequencing primers, dNTPs, termination (deaza) mixes (mix G, mix A, mix T, mix C), DTT solution, and sequencing buffers.

The kit optionally includes instructions for using the kit. The kit can also be optionally provided with instructions for in vitro transcription of the amplified cDNA molecules and with instructions for labeling and hybridizing the in vitro transcription products to microarrays. The kit can also be provided with instructions for labeling and/or sequencing. The kit can also be provided with instructions for cloning the PCR products into an expression vector to generate an expression library representative of the transcriptome of the sample at the time the sample was taken.

### IV. Definition of Chemical Terminology

When used in the context of a chemical group, “hydrogen” means —H; “hydroxy” means —OH; “oxo” means ═O; “halo” means independently —F, —Cl, —Br or —I; “amino” means —NH₂; “hydroxyamino” means —NHOH; “nitro” means —NO₂; imino means ═NH; “cyano” means —CN; “azido” means —N₃; in a monovalent context “phosphate” means —OP(O)(OH)₂ or a deprotonated form thereof, in a divalent context “phosphate” means —OP(O)(OH)O— or a deprotonated form thereof, “mercapto” means —SH; “thio” means ═S; “thioether” means —S—; “sulfonamido” means —NHS(O)₂—; “sulfonyl” means —S(O)₂—; “sulfinyl” means —S(O)—; “silyl” means —SiH₃.

For the groups described herein, the following parenthetical subscripts further define the groups as follows: “(Cn)” defines the number (n) of carbon atoms in the group. (Cn-n′) defines both the minimum (n) and maximum number (n′) of carbon atoms in the group. Similarly, “alkyl(C₂₋₁₀)” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CH₃(Me), —CH₂CH₃(Et), —CH₂CH₂CH₃(n-Pr), —CH(CH₃)₂(iso-Pr), —CH(CH₂)₂(cyclopropyl), —CH₂CH₂CH₂CH₃(n-Bu), —CH(CH₃)CH₂CH₃(sec-butyl), —CH₂CH(CH₃)₂(iso-butyl), —C(CH₃)₃(tert-butyl), —CH₂C(CH₃)₃(neo-pentyl), cyclobutyl, cyclopentyl, cyclohexyl, and cyclohexylmethyl are non-limiting examples of alkyl groups. The term “substituted alkyl” refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “aryl” when used without the “substituted” modifier refers to a monovalent group with an aromatic carbon atom as the point of attachment, said carbon atom forming part of one or more six-membered aromatic ring structure(s) wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃(ethylphenyl), —C₆H₄CH₂CH₂CH₃(propylphenyl), —C₆H₄CH(CH₃)₂, —C₆H₄CH(CH₂)₂, —C₆H₃(CH₃)CH₂CH₃(methylethylphenyl), —C₆H₄CH═CH₂(vinylphenyl), —C₆H₄CH═CHCH₃, naphthyl, and the monovalent group derived from biphenyl. The term “substituted aryl” refers to a monovalent group with an aromatic carbon atom as the point of attachment, said carbon atom forming part of one or more six-membered aromatic ring structure(s) wherein the ring atoms are all carbon, and wherein the monovalent group further has at least one atom independently selected from the group consisting of N, O, F, Cl, Br, I, Si, P, and S.

The term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). The term “substituted heteroaryl” refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non aromatic sulfur F, Cl, Br, I, Si, and P.

The term “acyl” when used without the “substituted” modifier refers to a monovalent group with a carbon atom of a carbonyl group as the point of attachment, further having a linear or branched, cyclo, cyclic or acyclic structure, further having no additional atoms that are not carbon or hydrogen, beyond the oxygen atom of the carbonyl group. The groups, —CHO, —C(O)CH₃(acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, —COC₆H₄CH₂CH₃, —COC₆H₃(CH₃)₂, and —C(O)CH₂C₆H₅, are non-limiting examples of acyl groups. The term “acyl” therefore encompasses, but is not limited to groups sometimes referred to as “alkyl carbonyl” and “aryl carbonyl” groups. The term “substituted acyl” refers to a monovalent group with a carbon atom of a carbonyl group as the point of attachment, further having a linear or branched, cyclo, cyclic or acyclic structure, further having at least one atom, in addition to the oxygen of the carbonyl group, independently selected from the group consisting of N, O, F, Cl, Br, I, Si, P, and S. The term “substituted acyl” encompasses, but is not limited to, “heteroaryl carbonyl” groups.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O-cyclohexyl. The term “substituted alkoxy” refers to the group —OR, in which R is a substituted alkyl, as that term is defined above.

Similarly, the terms “alkenyloxy”, “alkynyloxy”, “aryloxy”, “aralkoxy”, “heteroaryloxy”, “heteroaralkoxy” and “acyloxy”, when used without the “substituted” modifier, refers to groups, defined as —OR, in which R is alkenyl, alkynyl, aryl, aralkyl, heteroaryl, heteroaralkyl and acyl, respectively, as those terms are defined above. When any of the terms alkenyloxy, alkynyloxy, aryloxy, aralkyloxy and acyloxy is modified by “substituted,” it refers to the group —OR, in which R is substituted alkenyl, alkynyl, aryl, aralkyl, heteroaryl, heteroaralkyl and acyl, respectively.

The term “alkylamino” when used without the “substituted” modifier refers to the group —NHR, in which R is an alkyl, as that term is defined above. Non-limiting examples of alkylamino groups include: —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —NH-cyclopentyl, and —NH-cyclohexyl. The term “substituted alkylamino” refers to the group —NHR, in which R is a substituted alkyl, as that term is defined above.

The term “amido” (acylamino), when used without the “substituted” modifier, refers to the group —NHR, in which R is acyl, as that term is defined above. A non-limiting example of an acylamino group is —NHC(O)CH₃. When the term amido is used with the “substituted” modifier, it refers to groups, defined as —NHR, in which R is substituted acyl, as that term is defined above. The groups —NHC(O)OCH₃ and —NHC(O)NHCH₃ are non-limiting examples of substituted amido groups.

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Amplification of Hepatitis C Virus

Hepatitis C Virus (HCV) RNA was isolated from human serum samples using a commercially available kit (ToTALLY RNA, Ambion, Austin, Tex.). Reverse transcription of RNA was performed using a SuperScript kit (SuperScript III First-Strand Synthesis System for RT-PCR, Invitrogen, Carlsbad, Calif.), with gene specific primers (5′ AAC AGG AAA TGG CCT AAG AGG 3′ (SEQ ID NO:1), with the addition of 1 μM or 0.5 μM synthetic RNA oligonucleotides (5′NCCNCC3′) (SEQ ID NO:2), in which the 3′ hydroxyl group is blocked from extension by the addition of a 3 carbon alkyl group. PCR was conducted with a Phusion kit (Phusion Hot Start High Fidelity DNA Polymerase, New England Biolabs, Mass.), using 5 μl cDNA, 0.5 μM of HCV-specific primers (forward primer: 5′ TCA TGG TCG ACG GTC AGT AG 3′ (SEQ ID NO:3); reverse primer 5′ GGG GAG GAG GTA GAT GCC TA 3′) (SEQ ID NO:4), and 10 μl of 5× Phusion HF Buffer which contains 50 mM of MgCl₂, 10 mM dNTPs, and recombinant enzyme. PCR was done with DNA Thermal Cycler (Applied Biosystems Gene Amp PCR System 9700). Cycling conditions were as follows: denaturation at 98° C. for 10 s, annealing at 60° C. for 10 s, and elongation at 72° C. for 400 s.

### REFERENCES

The following references, to the extent that they provide exemplary procedural or other details supplementary to those set forth herein, are specifically incorporated herein by reference.
U.S. Pat. No. 5,508,178
U.S. Pat. No. 5,538,848
U.S. Pat. No. 5,723,591
U.S. Pat. No. 5,866,336
U.S. Pat. No. 5,876,930
U.S. Pat. No. 5,925,517
U.S. Pat. No. 5,958,700
U.S. Pat. No. 6,031,091
Birkenmeyer et al., J. Virolo. Meth., 35:117-126, 1991.
Compton, Nature, 350:91-92, 1991.
Davanloo et al., Proc. Natl. Acad. Sci. USA, 81:2035-2039, 1984.
Dietz et al., Mol. Gen. Genet., 221:283-286, 1990.
Fahy et al., PCR Meth. Appl., 1:25-33, 1991.
Guatelli et al., Proc. Nat. Acad. Sci. USA, 87:1874-1878, 1990.
Han et al., Protein Expr. Purif, 16:103-108, 1999.
Holland et al., Proc. Natl. Acad. Sci. USA, 88:7276-7280, 1991.
Landegren, Trends Genetics, 9:199-202, 1993.
Langmore, Pharmacogenomics, 3:557-560, 2002.
Liu et al., Cold Spring Harbor Protocols, Cold Spring Harbor, N.Y., 2008.
Sarin et al., Proc. Natl. Acad. Sci. USA, 85:7448-7451, 1988.
Stein et al., Nucl. Acids Res., 16:3209-3221, 1988.
Tanabe et al., Genes Chromo. Cancer, 38:168-176, 2003.
Telenius et al., Genomics, 13:718-725, 1992.
Walker, PCR Meth. Appl., 3:1-6, 1993.

1. A non-extendable nucleic acid for enhancing or increasing the yield of nucleic acid amplification or synthesis comprising a non-extendable nucleic acid of 5 or more nucleotides comprising a nucleotide sequence of ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analogs thereof, wherein x is any nucleotide or nucleotide analog.

2. The non-extendable nucleic acid of claim 1, wherein the non-extendable nucleic acid does not form a double stranded nucleic acid by either intra-oligonucleotide or inter-oligonucleotide hybridization at 20° C. or above.

3. The non-extendable nucleic acid of claim 1, further comprising a modified 3′ hydroxyl of the 3′ terminal nucleotide.

4. The non-extendable nucleic acid of claim 3, wherein an H, alkyl, arylalkyl, group replaces or is covalently attached to the 3′ hydroxyl group of the 3′ nucleotide.

5. The non-extendable nucleic acid of claim 1, further comprising a modified 5′ position of the 5′ nucleotide.

6. The non-extendable nucleic acid of claim 1, wherein the 5′ position comprises a mono-phosphate, a H, or an alkyl group.

7. The non-extendable nucleic acid of claim 1, wherein the oligonucleotide is an RNA oligonucleotide.

8. The non-extendable nucleic acid of claim 1, further comprising a detectable label.

9. The non-extendable nucleic acid of claim 8, wherein the detectable label is selected from the group consisting of fluorescers, chemiluminescers, dyes, biotin, haptens, enzymes, enzyme substrates, enzyme cofactors, enzyme inhibitors, enzyme subunits, metal ions, electron-dense reagents, and radioactive isotopes.

10. A method for amplifying a target nucleic acid sequence comprising:
contacting the target nucleotide sequence under hybridizing conditions with:
(a) an oligonucleotide primer;
(b) an amplification enhancer comprising a non-extendable oligonucleotide of 5 or more nucleotides comprising a nucleotide sequence of ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analogs thereof, wherein x is any nucleotide or nucleotide analog wherein x is any nucleotide or nucleotide analog; and
(c) an agent for polymerization of the nucleotides.

11. The method of claim 10, wherein the oligonucleotide does not form a double stranded oligonucleotide by either intra-oligonucleotide or inter-oligonucleotide hybridization at 20° C. or above

12. The method of claim 10, wherein the oligonucleotide is an RNA or an RNA analog.

13. The method of claim 10, further comprising a detectable label.

14. The method of claim 13, wherein the detectable label is selected from the group consisting of fluorescers, chemiluminescers, dyes, biotin, haptens, enzymes, enzyme substrates, enzyme cofactors, enzyme inhibitors, enzyme subunits, metal ions, electron-dense reagents, and radioactive isotopes.

15. The method of claim 10, wherein the target nucleic acid is a microbial DNA or RNA.

16. The method of claim 16, wherein the microbial DNA or RNA is a viral DNA or RNA.

17. The method of claim 10, wherein the agent for polymerization is a DNA polymerase or a DNA ligase.

18. (canceled)

19. The method of claim 10, wherein the agent for polymerization is an RNA polymerase or an RNA reverse transcriptase.

20-25. (canceled)

26. An amplicon formed by amplifying a nucleic acid in the presence of a non-extendable oligonucleotide of 5 or more nucleotides comprising a nucleotide sequence of ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analogs thereof, wherein x is any nucleotide or nucleotide analog; wherein the oligonucleotide does not form a double stranded oligonucleotide by either intra-oligonucleotide or inter-oligonucleotide hybridization at 20° C. or above.

27. (canceled)

28. A kit for amplifying nucleic acids comprising a non-extendable oligonucleotide of 5 or more nucleotides comprising a nucleotide sequence of ggxgg, ccxcc, gcxcg, gcxcg, aaxaa, ttxtt, atxta, taxat, xggxgg, xccxcc, xgcxcg, xgcxcg, xaaxaa, xttxtt, xatxta, xtaxat, or nucleotide analogs thereof, wherein x is any nucleotide or nucleotide analog.

29-35. (canceled)
